(19) United States
(12) Patent Application Publication
(10) Pub. No.: US 2024/0358785 A1
(43) Pub. Date: Oct. 31, 2024

(54) DIETARY AND FOOD SUPPLEMENT COMPOSITION FOR WEIGHT LOSS AND APPETITE REDUCTION AND USE THEREOF

(71) Applicant: Livia Lemes ROSA, Coconut Creek, FL (US)

(72) Inventor: Livia Lemes ROSA, Coconut Creek, FL (US)

(21) Appl. No.: 18/606,798

(22) Filed: Mar. 15, 2024

Related U.S. Application Data
(60) Provisional application No. 63/462,799, filed on Apr. 28, 2023.

(51) Int. Cl. A61K 36/24; A61K 9/48; A61K 33/24; A61K 35/00; A61K 35/748; A61K 36/04; A61K 36/185; A61K 36/537; A61K 36/55; A61K 36/68; A61K 36/82; A61P 3/04

(52) U.S. Cl. CPC A61K 36/24; A61K 9/4825; A61K 33/24; A61K 35/748; A61K 36/04; A61K 36/185; A61K 36/537; A61K 36/55; A61K 36/68; A61K 36/82; A61P 3/04; A61K 2035/115

(57) ABSTRACT

The present application pertains to the field of diet supplementation. In particular, the present application refers to a food supplement or dietary supplement composition with enhance weight loss and appetite reduction properties and use thereof. Said composition comprises a combination of natural ingredients which may be selected from a group consisting at least one of: linseed, green tea, chromium, spirulina, hibiscus, psyllium, glucomannan, agar-agar, chia seed, Caralluma.

### CROSS-REFERENCE TO RELATED APPLICATION

This application claims the benefit of U.S. Provisional Patent Application Ser. No. 63/462,799 entitled “DIETARY AND FOOD SUPPLEMENT COMPOSITION FOR WEIGHT LOSS AND APPETITE REDUCTION” and filed Apr. 28, 2023, the entirety of which is hereby incorporated herein by reference.

### BACKGROUND OF THE INVENTION

The present application pertains to the field of diet supplementation. In particular, the present application refers to a food supplement or dietary supplement composition with enhance weight loss and appetite reduction properties and use thereof.

This application also concerns a dietary supplement or dietary supplement composition that reduces hunger and promotes the maintenance of lean muscle mass.

The genesis of obesity and eating behavior involves appetite (hunger and satiety) and is coordinated by the activity of the Peripheral and Central Nervous Systems. More specifically, the regulatory site for the biochemistry of human eating behavior is the hypothalamus; this regulation occurs through meals that generate a feeling of gastric fullness.

The regulation of food intake and energy expenditure is carried out by a specific nucleus present in the hypothalamus and by a group of neuropeptides—such as neuropeptide Y—that interact with hormones like insulin, leptin, and ghrelin.

Leptin is a protein produced proportionally to the amount of adipose tissue, considered the satiety hormone, and participates in the neuroendocrine control of food intake, glycemic and lipid metabolism, and increased energy expenditure. On the other hand, ghrelin is a hormone secreted mainly by gastrointestinal tract cells and signals food intake.

Obesity is related, among other factors, to an imbalance in energy balance. In other words, it can result from an imbalance between food intake and energy expenditure.

Current available dietary supplements for weight loss and appetite reduction are known to be very inefficient and toxic to the human organism. Many of those of such rely heavily in synthetic chemical compounds which usually induce unwanted side effects and toxicity.

As an alternative, individuals seeking to lose weight or reduce their appetite may resort to exercise routine and bootcamps. However, such approaches are highly demandant and individuals usually drop out before completing the program.

Surgical intervention is another possible route. Suffice to say that these procedures, although effective, impose considerable risks to the patient and demand a strict diet routine afterwards.

Considering all the above, it is clear that the diet supplementation industry needs new easy to adhere, natural, and non-toxic solutions allowing for an efficient weight loss and appetite reduction.

Currently, the available natural supplements with thermogenic action promote the burning of body fat but are not directly associated with appetite reduction, satiety, and consequent weight loss. Among the most commonly used supplements, there are those based on chromium picolinate. However, the regulatory allowed amount of this compound is not sufficient to allow a significant decrease in hunger and food cravings.

Considering the inefficiency of currently marketed dietary supplements, the present invention involves a combination of natural extracts and fibers that not only provide satiety and consequent appetite reduction but also encourages the individual to consume more water daily.

### SUMMARY OF THE INVENTION

The present invention refers to a dietary or food supplement composition for weight loss and appetite reduction. Said composition comprises a combination of natural ingredients which may be selected from a group consisting of: linseed, green tea, chromium, spirulina, hibiscus, psyllium, glucomannan, agar-agar, chia seed, Caralluma.

An objective of the present invention is to provide a dietary and dietary supplement composition as a prophylactic method to prevent obesity and aid weight reduction. The mentioned dietary and dietary supplement composition is intended for human consumption and preferably administered orally. This composition is also indicated for obese individuals, meaning individuals with a BMI above 30.

### BRIEF DESCRIPTION OF THE INVENTION

The present invention achieves its objectives through a dietary composition comprising Carallumafimbriata with at least one of the following ingredients: linseed, green tea, trivalent chromium, spirulina, hibiscus, psyllium, glucomannan, agar-agar and chia seed.

According to one embodiment, said composition comprises between 1% and 50%, preferably between 5% and 40%, more preferably between 10% and 30%, and even more preferably between 15% and 25% by weight of Carallumafimbriata.

According to another embodiment, the said composition is still for oral administration.

In another embodiment, said composition is provided in the form of a capsule or tablet.

In another embodiment, said composition is provided in the form of a hard gelatin capsule size 1.

In another embodiment, said composition is configured to be administered at least once a day.

In a further embodiment, the present invention achieves its objectives through a solid oral dosage form comprising the dietary composition of the present invention.

In still yet a further embodiment, the present invention achieves its objectives through the use of the composition for weight and appetite reduction of the present invention.

### DETAILED DESCRIPTION OF THE INVENTION

The present invention refers to a dietary or food supplement composition for weight loss and appetite reduction. Said composition comprises a combination of natural ingredients that may have a synergistic effect and which may be selected from a group consisting of: linseed, green tea, chromium, spirulina, hibiscus, psyllium, glucomannan, agar-agar, chia seed, Caralluma. Furthermore, this composition is formulated in suitable carriers for oral administration.

The present invention includes several components that provide significant amounts of fiber to the composition. These fibers not only improve motility in the gastrointestinal tract but also promote a feeling of satiety and appetite reduction, leading the patient to consume larger volumes of water, resulting in a feeling of satiety throughout the day. This feeling of satiety is due to the release of ATP in the hypothalamus, which signals a state of sufficient energy, and that food intake is no longer necessary.

The feeling of satiety also reduces the production of the hormones ghrelin and leptin, preventing the formation of the hunger-promoting molecule, neuropeptide Y (NPY). This neuropeptide also blocks the action of the enzyme ATP-citrate lyase and hinders the formation of building blocks for fat production, such as acetyl-CoA and malonyl-CoA.

Moreover, some ingredients in the present invention contain glycosides that support the development of lean muscle mass. It is scientifically known that a higher rate of muscle mass promotes greater energy expenditure in an individual at rest and suppresses the sensory mechanism of hunger.

Additionally, the present invention comprises a composition provided in suitable solid vehicles for oral administration. This form of administration presents several advantages that contribute to achieving the objectives of the present invention, such as ease of administration, being a non-invasive method, greater chemical and physical stability, among others.

Particularly, the present invention is formulated in capsules, preferably in hard gelatin capsules or soft gelatin capsules. Moreover, the mentioned capsules have size 2, preferably size 1, more preferably size 0, and even more preferably size 00.

These gelatin capsules can be of conventional release, immediate-release, delayed-release, or extended-release types. More preferably, the present invention utilizes hard gelatin capsules with conventional release as the solid oral pharmaceutical form.

The powders of the components comprising the dietary composition disclosed by the present invention are mixed through dry blending in a V-blender until homogeneity is achieved. Subsequently, the complete filling of the powder is carried out using a capsule filling machine, and capsule counting is performed with a capsule counter.

Within the scope of the present invention, it should be understood by a skilled person that the components used in the composition of the present invention should be present in any quantity that provides the effects of appetite reduction, feeling of satiety, and consequent weight loss.

For example, in one aspect of the present invention, the amount of fine flaxseed powder present is between 0.1% and 20%, preferably between 0.5% and 15%, more preferably between 0.7% and 12%, and even more preferably between 1% and 10%.

In another aspect of the present invention, the amount of fine green tea powder present in the composition is between 0.1% and 20%, preferably between 0.5% and 15%, more preferably between 0.7% and 12%, and even more preferably between 1% and 10%.

In another aspect of the present invention, the amount of chromium (Cr3+) present in the composition is between 1% and 50%, preferably between 2% and 40%, more preferably between 2.5% and 30%, and even more preferably between 3% and 30%.

In another aspect of the present invention, the amount of spirulina present in the composition is between 1% and 15%, preferably between 2% and 13%, more preferably between 5% and 12%, and even more preferably between 7% and 10%.

In another aspect of the present invention, the amount of fine hibiscus powder present in the composition is between 0.1% and 20%, preferably between 0.5% and 15%, more preferably between 0.7% and 12%, and even more preferably between 1% and 10%.

In another aspect of the present invention, the amount of fine psyllium powder in the composition is between 0.1% and 20%, preferably between 0.5% and 15%, more preferably between 0.7% and 12%, and even more preferably between 1% and 10%.

In another aspect of the present invention, the amount of fine glucomannan powder in the composition is between 7% and 50%, preferably between 8% and 40%, more preferably between 12% and 35%, and even more preferably between 14% and 30%.

In another aspect of the present invention, the amount of agar-agar in the composition is between 1% and 50%, preferably between 2% and 30%, more preferably between 5% and 20%, and even more preferably between 7% and 15%.

In another aspect of the present invention, the amount of fine chia seed powder in the composition is between 1% and 50%, preferably between 2% and 30%, more preferably between 5% and 20%, and even more preferably between 7% and 15%.

In another aspect of the present invention, the amount of fine Caralluma fimbriata powder in the composition is between 1% and 50%, preferably between 5% and 40%, more preferably between 10% and 30%, and even more preferably between 15% and 25%.

According to one embodiment of the present invention, the dietary composition is defined and indicated below:

TABLE 1
Diet supplement composition example:

| Ingredient | Amount | |
|---|---|---|
| linseed | 50 | mg |
| green tea | 50 | mg |
| chromium | 35 | mcg |
| spirulina | 50 | mg |
| hibiscus | 50 | mg |
| psyllium | 70 | mg |
| glucomannan | 100 | mg |
| agar-agar | 70 | mg |
| chia seed | 50 | mg |
| Caralluma | 150 | mg |
| TOTAL | 740 | mg |

In a possible embodiment, the present composition may be prepared by dry mixing the all the ingredients until the mixture becomes a homogenous powder. The resulting powder may be added to capsules or larger recipients, such as bottles and cans.

The ingredient combination disclosed by the present application is a result of an extensive research and refined combination of different natural properties from each ingredient. The specific properties of each ingredient are described below.

The linseed, also known as flaxseed, belongs to the Linaceae family. Its fibers act in the release of glucose in the blood and reduce the accumulation of fat in the body—and it is also a gluten-free cereal alternative, which can be consumed by people with celiac disease. Consumption of linseed with other foods creates a barrier in the food bolus, which makes the release of glucose into the bloodstream much slower. This way, there are no glycemic spikes and less insulin, the hormone responsible for fat storage, needs to be used. When we have a lot of insulin circulating through the body, we quickly feel the need to consume more foods that will bring glucose to our body quickly, in this case foods like bread, cakes, cookies and sweets, which make you fat.

In addition, flaxseed helps balance the amounts of omega 3 and 6, which are polyunsaturated fats that act by reducing LDL cholesterol levels (bad) and increasing HDL (good cholesterol).

According to a preferred configuration of the present invention, the amount of fine flaxseed powder added to the composition is between 1 mg and 1000 mg, preferably between 10 mg and 500 mg, more preferably between 15 mg and 200 mg, and even more preferably between 20 mg and 100 mg.

The green tea botanica name is Camellia sinensis (L) O. Kuntze, belonging to the family Theaceae (Terntroemiaceae). Preferably, the dietary composition of the present inventio utilized only the leafs of said plant.

The green tea has lipolytic and diuretic action contains: proteins, carbohydrates, carbohydrates, ascorbic acid, B complex vitamins (thiamine—B1, riboflavin—B2 and B6), fluorine, betacarotene, folic acid, theanine, vitamin K, purine bases especially caffeine, theobromine and theophylline, essential oil; polyphenols: monosides of flavonols and flavones, catechols and epicatechins free and esterified by gallic acid and condensation products (catechins: epicatechin-EC, epigallocatechin-EGC, gallate-3-epicatechin-ECG, gallate-3-epigallocatechin-EGCG), and tannins.

The green tea helps dietary regimes, as it greatly accelerates metabolism, which makes it contraindicated for patients with heart problems.

According to a preferred configuration of the present invention, the amount of fine green tea powder added to the composition is between 1 mg and 1000 mg, preferably between 10 mg and 500 mg, more preferably between 15 mg and 200 mg, and even more preferably between 20 mg and 100 mg.

The chromium ingredient refers to trivalent chromium (Cr³⁺), which is considered an essential element. The trivalent chromium is derived, in the present composition, from compounds such as chromium picolinate, which could be added to the composition according an amount between 10 μg and 50 μg.

The nutritional supplementation of chromium in this form is considered a weight loss agent, as it makes the body use more carbohydrates as a source of energy and absorb more proteins, helping to reduce blood glucose, gain muscle mass and lose weight, mainly in the abdominal region.

According to a preferred configuration of the present invention, the amount of chromium added to the composition is between 1 mg and 1000 mg, preferably between 5 mg and 500 mg, more preferably between 10 mg and 200 mg, and even more preferably between 15 mg and 50 mg.

Spirulina is a microscopic, filamentous cyanobacterium, which derives its name from the helix or spiral structure of its filaments. Spirulina refers to the biomass of Arthrospira platensis, a bacteria found worldwide in blue-green seaweed. Spirulina is widely studied for use in human food because of its nutritional profile that makes it ideal as a food supplement, as it satisfactorily replaces artificial sources of nutrients, as it combines different constituents in a balanced way. These nutrients include protein, B-complex vitamins, minerals, high-quality proteins, antioxidants β-carotene and vitamin E, and polyunsaturated fatty acids, especially gama-linolenic acid.

Spirulina acts as an appetite suppressant, coating the stomach walls and promoting a feeling of gastric fullness and satiety, helping in the weight loss process. It has a supporting function in the supplementation of vegetarian individuals or in the treatment of anemia, due to the high content of vitamin B12; can be used as a dietary supplement in individuals with osteoporosis due to its mineral content. Improves intestinal transit and has a high digestive level, with an absorption close to 90%.

According to a preferred configuration of the present invention, the amount of spirulina added to the composition is between 1 mg and 100 mg, preferably between 5 mg and 90 mg, more preferably between 10 mg and 80 mg, and even more preferably between 20 mg and 70 mg.

The ingredient hibiscus has the power to stimulate fat burning, improve digestion, regularize the intestine and still acts in the fight against liquid retention. It also helps regulate appetite, as it has a certain influence on some hormones linked to this process, such as ghrelin and leptin. It is able to inhibit an enzyme called amylase, which is responsible for transforming starch into sugar, reduces the maturation process of cells that store fat in the body. So, with the reduction of the process, consequently there is a reduction of the chances of accumulating fat specifically in the abdomen and hips.

According to a preferred configuration of the present invention, the amount of fine hibiscus powder added to the composition is between 1 mg and 1000 mg, preferably between 10 mg and 500 mg, more preferably between 15 mg and 200 mg, and even more preferably between 20 mg and 100 mg.

Psyllium is a soluble fiber extracted from the seed husk of a plant called Plantago ovata. This fiber has the ability to increase up to 20 times in size when it comes into contact with other liquids, as it has high water absorption. For this reason, psyllium has a regulating effect on intestinal activity, increasing the volume and moisture of the stool and stimulating bowel movements. In addition, its fibers help control blood glucose, triglycerides and cholesterol and blood pressure, in addition to increasing satiety.

According to a preferred configuration of the present invention, the amount of fine psyllium powder added to the composition is between 1 mg and 5000 mg, preferably between 3 mg and 2000 mg, more preferably between 5 mg and 500 mg, and even more preferably between 10 mg and 100 mg.

Glucomannan is extracted from the root of Konjac, a medicinal plant known scientifically as Amorphophallus konjac, it is a vegetable fiber that helps you lose weight because it forms a type of gel in the stomach, increasing food digestion time and reducing hunger between meals. It also helps fight constipation because it promotes hydration and softness of the stool, facilitating bowel movements.

According to a preferred configuration of the present invention, the amount of fine glucomannan powder added to the composition is between 5 mg and 4000 mg, preferably between 10 mg and 1000 mg, more preferably between 15 mg and 500 mg, and even more preferably between 20 mg and 150 mg.

Agar-agar is a microalgae that has thickening, gelling and stabilizing properties. Due to its properties and the fact that it is rich in fiber and minerals, such as phosphorus, potassium, iron, chlorine and iodine, cellulose and proteins, agar-agar can help with the weight loss process and regulate bowel function. Agar-agar regulates the absorption of carbohydrates by the intestine, which helps to reduce blood glucose levels and insulin resistance and can be used by people with diabetes or who have glucose intolerance.

According to a preferred configuration of the present invention, the amount of agar-agar added to the composition is between 1 mg and 1000 mg, preferably between 10 mg and 500 mg, more preferably between 15 mg and 200 mg, and even more preferably between 20 mg and 100 mg.

Chia is a plant comprising seeds with fiber, protein and other nutrients that help you lose weight and detoxify. Its fibers have the ability to absorb a lot of water, turning into a kind of gel providing satiety.

Chia contains significant amounts of omega 3 and many studies have linked the consumption of this fatty acid with a decrease in inflammation, which would be interesting to reduce and prevent cellulite, an inflammatory process in the body. It also contains vitamin A, a nutrient that acts as an antioxidant against free radicals and helps reduce acne and prevent skin dryness. The seed also contains vitamin B2, which is important for the health of the skin, nails, and hair.

According to a preferred configuration of the present invention, the amount of fine chia seed powder added to the composition is between 5 mg and 1200 mg, preferably between 10 mg and 500 mg, more preferably between 15 mg and 250 mg, and even more preferably between 20 mg and 170 mg.

Caralluma fimbriata, also known as Karallamu or Shindala makadi, is an edible cactus used especially by people in India to reduce appetite and increase energy.

Caralluma acts as a natural appetite suppressant. The succulent is thought to block the effects of certain enzymes that play a role in fat formation. This forces the body to burn fat stores, potentially leading to weight loss. The main phytochemical constituents of the herb are pregnane glycosides, flavone glycosides, megastigma glycosides and saponins that block the fat-forming enzyme citrate lyase, supporting the development of lean muscle mass, which burns more fat at rest. These glycosides also suppress the hunger sensory mechanism found within the hypothalamus.

In compositions found in the prior art, Caralluma fimbriata is used individually or in combinations that only allow short-term action. However, in the present invention, the combination of Caralluma with the other raw materials disclosed in this invention allows the effects of the phytochemicals present in this herb to have a prolonged action.

According to a preferred configuration of the present invention, the amount of fine Carallumafimbriata powder added to the composition is between 10 mg and 2000 mg, preferably between 25 mg and 1200 mg, more preferably between 40 mg and 500 mg, and even more preferably between 50 mg and 250 mg.

According to a configuration of the present invention, the hard gelatin capsules containing the disclosed composition are stored in bottles. Preferably, the capsules are stored in opaque white bottles.

According to the present invention the capsule containing the final composition weights between 300 and 800 mg, more preferably between 400 and 700, more preferably 675 mg.

### EXAMPLES

In an experimental study of the dietary supplement disclosed by the present invention, participants (n=20) weighing between 64 kg and 120 kg ingested one capsule per day, preferably fasting in the morning, for 4 weeks. The results demonstrated that the composition disclosed by the present invention was effective in reducing weight. The patients lost between 8 kg and 12 kg during the study, with an average weight loss of 9.55 kg. The study also observed that participants who used the supplemental composition of the present invention along with regular physical exercise lost more weight compared to participants who only used the supplemental composition. Additionally, patients who engaged in exercise during the study period showed greater development of lean muscle mass aided by the glycosides present in some components of the formulation.

The examples ingredient listed, and their specific amount is not restricted to the example given in Table 1. Any person skilled in the art would know how to adjust the dietary supplement composition disclosed for the purpose of specific uses and particular individual needs.

Furthermore, some examples of possible embodiments of the present invention will be presented. These examples are variations of the component quantities described in this report, and the scope of protection of the present invention is limited to the claims.

### Example 1

Hard gelatin capsule size 00: Composition formulated for oral administration in a single daily dose.

| Ingredient | Amount |
|---|---|
| linseed | 70 mg |
| green tea | 70 mg |
| chromium | 60 mg |
| spirulina | 50 mg |
| hibiscus | 30 mg |
| psyllium | 75 mg |
| glucomannan | 50 mg |
| agar-agar | 50 mg |
| chia seed | 50 mg |
| Caralluma | 170 mg |
| TOTAL | 675 mg |

### Example 2

Hard gelatin capsule size 0: Composition formulated for oral administration twice daily.

| Ingredient | Amount |
|---|---|
| linseed | 30 mg |
| green tea | 40 mg |
| chromium | 30 mg |
| spirulina | 40 mg |
| hibiscus | 30 mg |
| psyllium | 40 mg |
| glucomannan | 50 mg |
| agar-agar | 50 mg |
| chia seed | 30 mg |
| Caralluma | 60 mg |
| TOTAL | 400 mg |

### Example 3

Hard gelatin capsule size 1: Composition formulated for oral administration 1 to 3 times a day.

| Ingredient | Amount |
|---|---|
| linseed | 20 mg |
| green tea | 20 mg |
| chromium | 15 mg |
| spirulina | 20 mg |
| hibiscus | 20 mg |
| psyllium | 20 mg |
| glucomannan | 50 mg |
| agar-agar | 20 mg |
| chia seed | 20 mg |
| Caralluma | 95 mg |
| TOTAL | 300 mg |

1. A dietary supplement composition comprising Carallumafimbriata with at least one of the following ingredients: flaxseed, green tea, trivalent chromium, spirulina, hibiscus, psyllium, glucomannan, agar-agar, and chia seed.

2. The dietary supplement composition according to claim 1, wherein the said composition comprises between 1% and 50%, preferably between 5% and 40%, more preferably between 10% and 30%, and even more preferably between 15% and 25% by weight of Caralluma fimbriata.

3. The dietary supplement composition, according to claim 1, wherein the said composition is for oral administration.

4. The dietary supplement composition, according claim 1, wherein the said composition is provided in the form of a capsule or tablet.

5. The dietary supplement composition according to claim 4, wherein the said composition is provided in the form of a hard gelatin capsule size 1.

6. The dietary supplement composition according to claim 4, wherein the said composition is configured to be administered at least once a day.

7. A solid oral dosage form comprising the dietary supplement composition according to claim 1.

8. Use of the composition, according to claim 1, wherein said composition is for weight and appetite reduction.
